SEQUENCE LISTING (16 sequences)

SEQ ID NO: 1 - protein, 107 aa, Artificial Sequence
  The amino acid sequence of the light chain variable region of the CD19 antibody
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 2 - protein, 122 aa, Artificial Sequence
  The amino acid sequence of the heavy chain variable region of the CD19 antibody
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 3 - protein, 236 aa, Artificial Sequence
  The LC sequence of the Her2 antibody
MDMRVPAQLLGLLLLWLRGARCDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 237 aa, Artificial Sequence
  The HC sequence of the Her2 antibody
MEFGLSWVFLVAILKGVQCEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 5 - protein, 119 aa, Artificial Sequence
  The VH sequence of the scFv of the EGFR antibody
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 6 - protein, 107 aa, Artificial Sequence
  The VL sequence of the EGFR antibody
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 7 - protein, 121 aa, Artificial Sequence
  The VH sequence of the PSMA antibody
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSS

SEQ ID NO: 8 - protein, 107 aa, Artificial Sequence
  The VL sequence of the PSMA antibody
DIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGGGTKLEIK

SEQ ID NO: 9 - protein, 450 aa, Artificial Sequence
  The HC sequence of the PDL1 antibody
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 216 aa, Artificial Sequence
  The light chain sequence of the PDL1 antibody
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 11 - protein, 119 aa, Artificial Sequence
  The VH sequence of the CD3 antibody
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 12 - protein, 106 aa, Artificial Sequence
  The VL sequence of the CD3 antibody
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 13 - protein, 125 aa, Artificial Sequence
  The VH sequence of the CD3 antibody
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 14 - protein, 110 aa, Artificial Sequence
  The VL sequence of the CD3 antibody
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLA

SEQ ID NO: 15 - protein, 113 aa, Artificial Sequence [sequence omitted]

SEQ ID NO: 16 - protein, 111 aa, Artificial Sequence
  The VL sequence of the PD-1 antibody
LEMAEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIK

(19) United States
(12) Patent Application Publication
(10) Pub. No.: US 2022/0257786 A1
(43) Pub. Date: Aug. 18, 2022

(54) MULTICELLULAR TARGETING LIPOSOME

(71) Applicant: SHANGHAI JIAO TONG UNIVERSITY, Shanghai (CN)

(72) Inventors: Yuhong XU, Shanghai (CN); Fang XIE, Shanghai (CN)

(73) Assignee: SHANGHAI JIAO TONG UNIVERSITY, Shanghai (CN)

(21) Appl. No.: 16/646,577

(22) PCT Filed: Sep. 7, 2018
(86) PCT No.: PCT/CN2018/104533

§ 371 Date: Jul. 23, 2020

(30) Foreign Application Priority Data
Sep. 14, 2017 (CN) 201710828962.X

(51) Int. Cl. A61K 47/68; A61K 47/69; A61K 9/127; A61P 35/00; A61P 37/02

(52) U.S. Cl. CPC A61K 47/68; A61K 47/69; A61P 37/02; A61P 35/00; A61K 9/1271

(57) ABSTRACT

The present disclosure belongs to the field of the use of liposome preparations in the preparation of tumor drugs, and in particular, relates to the construction and use of a multicellular targeting liposome. Due to that a single liposome includes multiple antibodies against different cells, and a liposome with a stable structure and long circulation characteristics in vivo is constituted by molecular self-assembly, the multicellular targeting liposome can sequentially or simultaneously bind to multiple different cells by means of the action of antibodies to promote the identification of, communication between, the cytotoxicity of, the pro-apoptosis or clearance of different cells, etc.

### CROSS REFERENCES TO RELATED APPLICATIONS

This is a Sect. 371 National Stage application of a PCT International Application No. PCT/CN2018/104533, filed on Sep. 7, 2018, which claims priority of a Chinese Patent Applications No. 201710828962X, filed on Sep. 14, 2017, the content of which is hereby incorporated by reference in its entirety for all purposes.

### INCORPORATION BY REFERENCE OF SEQUENCE LISTING ON ASCII TEXT FILE

The content of the following submission on ASCII text file is incorporated herein by reference in its entirety: a computer readable form (CRF) of the Sequence Listing (file name: 200022-sequence-listing-ST25.txt, date recorded: Sep. 4, 2020, size: 24 KB).

### TECHNICAL FIELD

The present disclosure belongs to the technical field of pharmaceutical preparations, liposome drug carriers and antibody targeting, and particularly relates to a multicellular targeting liposome pharmaceutical preparation that acts simultaneously with immune effector cells and target cells.

### BACKGROUND

Liposome is a drug carrier with a bilayer vesicle-like structure based on phospholipid and/or cholesterol. Liposomes are artificially prepared bilayer vesicles formed by lipid molecules. Since the lipid molecule has a hydrophilic head and a hydrophobic tail, hydrophilic drugs can be contained in the internal aqueous phase, and hydrophobic drugs can be embedded in the lipid bilayer. In addition, liposomes have good histocompatibility, low toxicity and good biosecurity, therefore have attracted widespread attention and been applied in the field of drug delivery systems. In particular, the development of liposome targeting technology has endowed the lipid drug carrier with unique in vivo behavior, enabling it to enrich the target site and then interact with the target cell specifically. It is hoped that by optimizing the selection of targets and improving the structure and performance of targeting ligands, clinical efficacy of liposome drugs can be improved, including reducing drug system toxicity and increasing therapeutic index.

The research and development idea of targeting liposome drug delivery system is to design various types of active targeting ligands and drug carriers, targeting the receptors that are highly expressed or specifically expressed on the surface of tumor cells. The active targeting drugs developed according to the above design idea have more optimized targeting capability at the lesion site than the free drugs delivered without targeting carriers, for example, the drug concentration at the target site is increased, the pharmacodynamics is improved, etc. However, this type of targeting drug delivery system only acts on a specific type of tumor cells, it cannot effectively clear the tumor, and may induce the problems such as minimal residual disease and multidrug resistance of the tumor.

In recent years, the understanding of tumor heterogeneity is improved, therefore the mechanism of the limitation of single-cell targeting has been elucidated. For example, the differences in tumor molecular markers between different patients, the differences in molecular markers between different tumor cells within the same tumor in the same patient, and the different types of cells and the interactions within the same tumor. Aiming at the characteristics of tumor heterogeneity, the researchers have expanded the design idea of targeting delivery. For example, the double-target liposome can enhance the binding ability between the carrier and the cell, and promote the uptake of drugs by the cell. Drug carriers targeting tumor-related cells can inhibit tumor growth through other mechanisms, such as acting on tumor new blood vessels, fibroblasts, and tumor stem cells.

Although such targeting delivery technology has improved therapeutic, the design idea still resides within the established framework of single-cell targeting. There are a wide range of dynamic interactions between different cells within the tumor, such as lymphocyte infiltration, macrophage phagocytosis, antigen presentation, and intercellular communication based on cytokines and exosomes. These interactions include immune surveillance and clearance to inhibit tumor growth, as well as the microenvironment that promotes tumor growth. Moreover, blocking or promoting these intercellular interactions has proven to be feasible and effective in clinical practice, such as vaccine therapy based on antigen-specific T cells, antibody therapy that relies on the cytotoxic effect of NK cell, and immune checkpoint inhibitor therapy for blocking tumor from immune escaping. In contrast, single-cell targeting drug delivery systems can only regulate physiological processes by aiming at specific target cells, but cannot utilize the intercellular interactions in tumor tissues to achieve effective treatment.

Therefore, in the present disclosure, antibodies against different cells are modified on a single liposome. The liposome with a stable structure and long circulation characteristics in vivo is constituted by molecular self-assembly. The liposome can sequentially or simultaneously bind to multiple different cells by means of the action of antibodies, to realize the blocking or promoting of intercellular interactions, so as to promote the recognition, communication, the cytotoxicity, the pro-apoptosis or clearance of different cells, etc. In addition, targeting drug delivery for multiple cells is also achieved by lipid drug carriers, which avoids problems such as drug interactions that may occur in combination therapy, and improves the pharmacological effects.

### SUMMARY

The present disclosure provides a multicellular targeting liposome pharmaceutical preparation capable of sequentially or simultaneously binding to multiple different cells, and preparation and application thereof.

The objects of the present disclosure are achieved by the following technical solutions:

A first aspect of the present disclosure provides a multicellular targeting liposome. The multicellular targeting liposome includes a liposome, a predominant antibody and an auxiliary antibody modified on the surface of the liposome, the predominant antibody specifically binds to target molecules on the surface of the target cells, and the auxiliary antibody specifically binds to immune effector cells.

Further, the multicellular targeting liposome is capable of simultaneously or sequentially binding to immune effector cells and target cells, such that the immune effector cells recognize target cells and activate immune effects.

Further, the molar ratio of the predominant antibody to the auxiliary antibody on each liposome is between 100/1 and 1/1.

Further, each liposome has 1-1000 predominant antibodies and 1-1000 auxiliary antibodies on the surface.

Further, the predominant antibody and the auxiliary antibody are displayed on the surface of the liposome through covalent linkage or hydrophobic, hydrophilic interaction, respectively.

As exemplified in some embodiments of the present disclosure, the predominant antibody and the auxiliary antibody are respectively connected to a lipid molecule in the liposome. The lipid molecule has a maleimide group. The predominant antibody and the auxiliary antibody are respectively connected to the maleimide group of the lipid molecule. The predominant antibody and the auxiliary antibody are respectively connected to the lipid molecule via a thioether bond.

Further, the target cell is selected from a group consisting of a tumor cell, a microorganism, and a microorganism-infected cell. Therefore, the antigen targeted by the predominant antibodies is selected from a group consisting of a microbial antigen, a tumor-related antigen, a tumor cell surface specific antigen, a highly expressed antigen on the tumor cell surface, and a highly expressed antigen in a tumor tissue or a tumor blood vessel. That is, the target molecule is selected from a group consisting of a microbial antigen, a tumor-related antigen, a tumor cell surface specific antigen, a highly expressed antigen on the tumor cell surface, and a highly expressed antigen in a tumor tissue or a tumor blood vessel.

Further, the specific target molecule of the predominant antibody is selected from a group consisting of CD19, CD20, PSMA, Her2/neu, EGFR, LGR5 or PDL1.

In some embodiments of the present disclosure, the specific target molecule of the predominant antibody is a CD19. The CD19 is a human CD19 antigen of human B lymphocytic leukemia or human B cell lymphoma.

Further, the immune effector cell is an effector cell capable of producing cytotoxicity, pro-apoptosis or clearance effects on target cells. The immune effector cell is selected from T lymphocyte, NK cell, NKT cell, macrophage, or neutrophil.

Therefore, the antigen targeted by the auxiliary antibody is selected from the antigen expressed by tumor cells, lymphocytes or myeloid cells. Therefore, the multicellular targeting liposome can simultaneously or sequentially bind to a tumor cell, a lymphocyte, or a myeloid cell. That is, the same liposome first binds to a tumor cell and then a lymphocyte, or vice versa.

In some embodiments of the present disclosure, the immune effector cell is a T lymphocyte.

In some embodiments of the present disclosure, the effector antigen of the immune effector cell is CD3, and the auxiliary antibody is a CD3 antibody.

Further, the auxiliary antibody is selected from a CD3 antibody and a fragment, a PD1 antibody and a fragment, a CTLA4 antibody and a fragment, a CD4OL antibody and a fragment.

The T lymphocyte may be an unactivated T cell, or an in-vitro activated or amplified T cell, for example, an antigen-specific T lymphocyte, a tumor infiltrating lymphocyte, a cytotoxic T cell, a helper T cell, a lymphokine-activated killer cell (LAK cells), a γδ T cell, a chimeric antigen receptor T cell, or a T cell receptor chimeric T cell.

The T lymphocyte is an unactivated T cell, or an in-vitro activated or amplified T cell.

The activation or amplification may be activation or amplification by antigen, antibody, polypeptide, polypeptide-maj or histocompatibility complex, small molecule, cytokine, immune checkpoint inhibitor or gene editing.

Further, the form of the predominant antibody and the auxiliary antibody is selected from IgG, Fab′ fragment, F(ab′)2 fragment, Fab fragment, single-chain Fv fragment, minibody, nanobody, unibody, or diabody.

As exemplified in some embodiments of the present disclosure, the antibody is a Fab′ fragment of an antibody, which is obtained after removing the Fc fragment and reducing the antibody. The antibody includes a light chain variable region and a heavy chain variable region.

As exemplified in some embodiments of the present disclosure, the predominant antibody is a Fab′ fragment of a CD19 antibody, and the amino acid sequence of the light chain variable region is shown in SEQ ID NO. 1, specifically:

DIQMTQTTSSLSASLGDRVTISCRASQDIRNYLNWYQQKPDGTVKLLIYYT
SRLHSGVPSKFSGSGSGTDYSLTISNLEQEDIATYFCQQGNTLPWTFAGGT
KLEIK.

The amino acid sequence of the heavy chain variable region is shown in SEQ ID NO. 2, specifically:

EVQLQQSGPELVKPGASMKISCKASGYSFTGYTMNWVKQSHGKNLEWMGLI
NPYKGVSTYNQKFKDKATLTVDKSSSTAYMELLSLTSEDSAVYYCARSGYY
GDSDWYFDVWGQGTTLTVFS.

In addition, the predominant antibody may be a Her2 antibody, and the LC sequence of the Her2 antibody is shown in SEQ ID NO. 3, specifically:

MDMRVPAQLLGLLLLWLRGARCDIQMTQSPSSLSASVGDRVTITCRASQDV
NTAVAWYQQKPGKAPKLLIYSASFLYSGVPSRFSGSRSGTDFTLTISSLQP
EDFATYYCQQHYTTPPTFGQGTKVEIKRTVAAPSVFIFPPSDEQLKSGTAS
VVCLLNNFYPREAKVQWKVDNALQSGNSQESVTEQDSKDSTYSLSSTLTLS
KADYEKHKVYACEVTHQGLSSPVTKSFNRGEC.

The HC sequence of the Her2 antibody is shown in SEQ ID NO. 4, specifically:

MEFGLSWVFLVAILKGVQCEVQLVESGGGLVQPGGSLRLSCAASGFNIKDT
YIHWVRQAPGKGLEWVARIYPTNGYTRYADSVKGRFTISADTSKNTAYLQM
NSLRAEDTAVYYCSRWGGDGFYAMDYWGQGTLVTVSSASTKGPSVFPLAPS
SKSTSGGTAALGCLVKDYFPEPVTVSWNSGALTSGVHTFPAVLQSSGLYSL
SSVVTVPSSSLGTQTYICNVNHKPSNTKVDKKV.

In addition, the predominant antibody may be an scFv fragment of an EGFR antibody, and the VH sequence of the scFv of the EGFR antibody is shown in SEQ ID NO. 5, specifically:

QVQLKQSGPGLVQPSQSLSITCTVSGFSLTNYGVHWVRQSPGKGLEWLGVI
WSGGNTDYNTPFTSRLSINKDNSKSQVFFKMNSLQSNDTAIYYCARALTYY
DYEFAYWGQGTLVTVSA.

The VL sequence of the EGFR antibody is shown in SEQ ID NO. 6, specifically:

DILLTQSPVILSVSPGERVSFSCRASQSIGTNIHWYQQRTNGSPRLLIKYA
SESISGIPSRFSGSGSGTDFTLSINSVESEDIADYYCQQNNNWPTTFGAGT
KLELK.

The predominant antibody may be a PSMA antibody, and the VH sequence of the PSMA antibody is shown in SEQ ID NO. 7, specifically:

QVQLVESGGGLVKPGESLRLSCAASGFTFSDYYMYWVRQAPGKGLEWVAII
SDGGYYTYYSDIIKGRFTISRDNAKNSLYLQMNSLKAEDTAVYYCARGFPL
LRHGAMDYWGQGTLVTVSS.

The VL sequence of the PSMA antibody is shown in SEQ ID NO. 8, specifically:

DIQMTQSPSSLSASVGDRVTITCKASQNVDTNVAWYQQKPGQAPKSLIYSA
SYRYSDVPSRFSGSASGTDFTLTISSVQSEDFATYYCQQYDSYPYTFGGGT
KLEIK.

The predominant antibody may be a PDL1 antibody, and the HC sequence of the PDL1 antibody is shown in SEQ ID NO. 9, specifically:

| | |
|---|---|
| EVQLLESGGG LVQPGGSLRL SCAASGFTFS SYIMMWVRQA PGKGLEWVSS | 50 |
| IYPSGGITFY ADTVKGRFTI SRDNSKNTLY LQMNSLRAED TAVYYCARIK | 100 |
| LGTVTTVDYW GQGTLVTVSS ASTKGPSVFP LAPSSKSTSG GTAALGCLVK | 150 |
| DYFPEPVTVS WNSGALTSGV HTFPAVLQSS GLYSLSSVVT VPSSSLGTQT | 200 |
| YICNVNHKPS NTKVDKKVEP KSCDKTHTCP PCPAPELLGG PSVFLFPPKP | 250 |
| KDTLMISRTP EVTCVVVDVS HEDPEVKFNW YVDGVEVHNA KTKPREEQYN | 300 |
| STYRVVSVLT VLHQDWLNGK EYKCKVSNKA LPAPIEKTIS KAKGQPREPQ | 350 |
| VYTLPPSRDE LTKNQVSLTC LVKGFYPSDI AVEWESNGQP ENNYKTTPPV | 400 |
| LDSDGSFFLY SKLTVDKSRW QQGNVFSCSV MHEALHNHYT QKSLSLSPGK | 450. |

The light chain sequence of the PDL1 antibody is shown in SEQ ID NO. 10, specifically:

| | |
|---|---|
| QSALTQPASV SGSPGQSITI SCTGTSSDVG GYNYVSWYQQ HPGKAPKLMI | 50 |
| YDVSNRPSGV SNRFSGSKSG NTASLTISGL QAEDEADYYC SSYTSSSTRV | 100 |
| FGTGTKVTVL GQPKANPTVT LFPPSSEELQ ANKATLVCLI SDFYPGAVTV | 150 |
| AWKADGSPVK AGVETTKPSK QSNNKYAASS YLSLTPEQWK SHRSYSCQVT | 200 |
| HEGSTVEKTV APTECS. | |

As exemplified in some embodiments of the present disclosure, the auxiliary antibody is a Fab′ fragment of a CD3 antibody, and the VH sequence of the CD3 antibody is shown in SEQ ID NO. 11, specifically:

DIKLQQSGAELARPGASVKMSCKTSGYTFTRYTMHWVKQRPGQGLEWIGYI
NPSRGYTNYNQKFKDKATLTTDKSSSTAYMQLSSLTSEDSAVYYCARYYDD
HYCLDYWGQGTTLTVSS.

The VL sequence of the CD3 antibody is shown in SEQ ID NO. 12, specifically:

DIQLTQSPAIMSASPGEKVTMTCRASSSVSYMNWYQQKSGTSPKRWIYDTS
KVASGVPYRFSGSGSGTSYSLTISSMEAEDAATYYCQQWSSNPLTFGAGTK
LELK.

Alternatively, the VH sequence of the CD3 antibody is shown in SEQ ID NO. 13, specifically:

EVQLVESGGGLVQPGGSLKLSCAASGFTFNKYAMNWVRQAPGKGLEWVARI
RSKYNNYATYYADSVKDRFTISRDDSKNTAYLQMNNLKTEDTAVYYCVRHG
NFGNSYISYWAYWGQGTLVTVSS.

The VL sequence of the CD3 antibody is shown in SEQ ID NO. 14, specifically:

QTVVTQEPSLTVSPGGTVTLTCGSSTGAVTSGNYPNWVQQKPGQAPRGLIG
GTKFLAPGTPARFSGSLLGGKAALTLSGVQPEDEAEYYCVLWYSNRWVFGG
GTKLTVLA.

The auxiliary antibody may also be a PD-1 antibody, and the VH sequence of the PD-1 antibody is shown in SEQ ID NO. 15, specifically:

QVQLVESGGGVVQPGRSLRLDCKASGITFSNSGMHWVRQAPGKGLEWVAVI
WYDGSKRYYADSVKGRFTISRDNSKNTLFLQMNSLRAEDTAVYYCATNDDY
WGQGTLVTVSS.

The VL sequence of the PD-1 antibody is shown in SEQ ID NO. 16, specifically:

LEMAEIVLTQSPATLSLSPGERATLSCRASQSVSSYLAWYQQKPGQAPRLL
IYDASNRATGIPARFSGSGSGTDFTLTISSLEPEDFAVYYCQQSSNWPRTF
GQGTKVEIK.

The present disclosure has no special limitation on the composition of the liposome, as long as it is a liposome. The components of the liposome may be selected from the group consisting of egg phospholipid, hydrogenated soybean phosphatidylcholine, hydrogenated egg phosphatidylcholine, dilauroylphosphatidylcholine, dimyristoylphosphatidylcholine, dipalmitoylphosphatidylcholine, distearoylphosphatidylcholine, 1-myristoyl-2-palmitoylphosphatidylcholine, 1-palmitoyl-2-stearoylphosphatidylcholine, 1-stearoyl-2-palmitoylphosphatidylcholine, 1-palmitoyl-2-oleylphosphatidylcholine, 1-stearoyl-2-linoleylphosphatidylcholine, dioleylphosphatidylcholine, hydrogenated dipalmitoylphospholipidcholine, distearoylphosphatidylcholine, dimyristoylphosphatidic acid, dimyristoylphosphatidic acid, dipalmitoylphosphatidic acid, dipalmitoylphosphatidic acid, distearoylphosphatidic acid, dimyristoyl phosphatidylethanolamine, dipalmitoylphosphatidylethanolamine, brain phosphatidylserine, dimyristoylphosphatidylserine, dipalmitoylphosphatidylserine, egg phosphatidylglycerol, dilauroylphosphatidyl glycerol, dimyristoylphosphatidyl glycerol, dipalmitoylphosphatidylglycerol, distearylphosphatidylglycerol, dioleoylphosphatidylglycerol, brain sphingomyelin, dipalmitoyl sphingomyelin or distearoyl sphingomyelin, cholesterol, dioleoxypropyltrimethylammonium chloride (DOTAP), dioleyl chloropropyl trimethylammonium chloride (DOTMA), dimethyldioctadecylammonium bromide (DDAB), dimethylaminoethane carbamoyl-cholesterol (DC-Chol), spermine-5-carboxyaminoacetic acid octacosylamide (DOGS), dioleosuccinylglycerol choline ester (DOSC), dioleyl chlorospermine carboxyl amide ethyl dimethyl propyl ammonium trifluoroacetate (DOSPA), or a combination of two or more thereof.

As exemplified in some embodiments of the present disclosure, the components of the liposome include phosphatidylcholine, cholesterol, a lipid linked to a predominant antibody, and a lipid molecule linked to an auxiliary antibody.

Further, the liposome may be a blank liposome or a drug-loading liposome.

A second aspect of the present disclosure provides a method for preparing the aforementioned multicellular targeting liposome. A post-insertion method is used. The method includes the following:

(1) connecting the predominant antibody and the auxiliary antibody with the lipid molecule respectively to obtain the predominant antibody-lipid molecule and the auxiliary antibody-lipid molecule;

(2) mixing the obtained predominant antibody-lipid molecule and the auxiliary antibody-lipid molecule with the constructed liposome, and incubating to obtain a mixed solution;

(3) dialyzing or ultrafiltrating the obtained mixed solution to remove unloaded antibody and antibody-lipid complexes, and obtaining the multicellular targeting liposome.

Preferably, the molar ratio of the predominant antibody to the lipid molecule is (0.1-5): 1000.

Preferably, the molar ratio of the auxiliary antibody to the lipid molecule is (0.1-5): 1000.

A third aspect of the present disclosure provides the use of the aforementioned multicellular targeting liposome in preparing immunotherapeutic drugs and antitumor drugs.

A fourth aspect of the present disclosure provides a method for treating tumors, including: administering the aforementioned multicellular targeting liposome to a tumor patient. The method may be in vitro. The method may be non-therapeutic. The tumor may be a tumor that is CD19-positive on the cell surface.

Compared with the traditional technology, the present disclosure has the following beneficial effects:

The multicellular targeting liposome of the present disclosure has a mechanism similar to that of the multi-targeting antibodies. The present disclosure further proposes to optimize effector cell activation effects on different target cells and target molecules by adjusting the density and relative proportions of the predominant antibody and auxiliary antibody. Furthermore, the effects of drugs loaded in the liposome on target cells and effector cells are further enhanced. Finally, the production, preparation and quality control of the multicellular liposome are also more feasible compared with multi-targeting antibodies.

### BRIEF DESCRIPTION OF THE DRAWINGS

FIG. 1A: Reduced SDS-PAGE images and non-reduced SDS-PAGE images of full-length CD3 antibody, purified anti-CD3 antibody F(ab′)2 fragment, anti-CD3 antibody Fab′ fragment-polymer-DSPE linked product, stained with Coomassie brilliant blue.

FIG. 1B: SDS-PAGE images of full-length CD19 antibody, purified anti-CD19 antibody F(ab′)2 fragment, anti-CD19 antibody Fab′ fragment-polymer-DSPE linker, stained with Coomassie brilliant blue.

FIG. 1C: SDS-PAGE images of anti-CD3 single-chain antibody (scFv) fragment, anti-CD3 single-chain antibody (scFv) fragment-polymer-DSPE linker, stained with Coomassie brilliant blue.

FIG. 2: Particle size distribution of multicellular targeting liposome, single-targeting liposome, and non-targeting liposome.

FIG. 3A: The specific binding of CD3 and CD19 multicellular targeting liposome and single-targeting liposome to target cells Jurkat.

FIG. 3B: The specific binding of CD3 and CD19 multicellular targeting liposome and single-targeting liposome to target cells Raji.

FIG. 4: Intercellular binding of CD3+ and CD19+ target cells mediated by multicellular targeting liposome, single-targeting liposome, and non-targeting liposome.

FIG. 5A: The cell-killing effect of the preactivated effector cells mediated by the multicellular targeting liposome on target cells Raji; the cytotoxicity 24 h after dosing is detected by the LDH method; the effector cells are T cells after activation and amplification by CD3 antibody and CD28 antibody for 3 days.

FIG. 5B: The cell-killing effect of the preactivated effector cells mediated by the multicellular targeting liposome on target cells Raji; the cytotoxicity 24 h after dosing is detected by the LDH method; the effector cells are LAK cells after activation and amplification by human IL-2.

FIG. 6A: The cell-killing effect of the preactivated effector cells mediated by the multicellular targeting liposome on target cells Raji, the effector cells are human γδT cells after amplification by zoledronic acid; the cytotoxicity 24 h after dosing is detected by LDH method; cell killing situation with different effector cell/target cell ratios.

FIG. 6B: The cell-killing effect of the preactivated effector cells mediated by the multicellular targeting liposome on target cells Raji, the effector cells are human γδT cells after amplification by zoledronic acid; the cytotoxicity 24 h after dosing is detected by LDH method; cell killing situation mediated by liposomes with different dose and targeting.

FIG. 6C: The cell-killing effect of the preactivated effector cells mediated by the multicellular targeting liposome on target cells Raji, the effector cells are human γδT cells after amplification by zoledronic acid; the cytotoxicity 24 h after dosing is detected by LDH method; cell killing situation mediated by liposomes with different dose and targeting.

FIG. 7A: The cell-killing effect of the unstimulated effector cells mediated by the multicellular targeting liposome on target cells Raji, the effector cells are unactivated human lymphocytes; the cytotoxicity 5 h after dosing is detected by PI staining method, i.e. the target cell killing situation after 5 h of dosing.

FIG. 7B: The cell-killing effect of the unstimulated effector cells mediated by the multicellular targeting liposome on target cells Raji, the effector cells are unactivated human lymphocytes; the cytotoxicity 24 h after dosing is detected by propidium iodide (PI) staining method, i.e. the target cell killing situation after 24 h of dosing.

FIG. 8A: The cell-killing effect of the unstimulated lymphocyte mediated by the multicellular targeting liposome on target cells Raji, the effector cells are unactivated human lymphocytes; cell killing situation with different effector cell/target cell ratios (PI staining method).

FIG. 8B: The cell-killing effect of the unstimulated lymphocyte mediated by the multicellular targeting liposome on target cells Raji, the effector cells are unactivated human lymphocytes; cell killing situation mediated by liposomes with different dose and targeting. (LDH method).

FIG. 8C: The cell-killing effect of the unstimulated lymphocyte mediated by the multicellular targeting liposome on target cells Raji, the effector cells are unactivated human lymphocytes; cell killing situation mediated by liposomes with different dose and targeting. (LDH method).

FIG. 9A: The cell-killing effect of the unstimulated lymphocyte mediated by the multicellular targeting liposome on target cells, the effector cells are unactivated lymphocytes, and the target cells are Raji cells; the cytotoxicity 5 h after dosing is detected by LDH method. The predominant antibody of the multicellular targeting liposome is a different clone of the anti-CD3 IgG monoclonal antibody (2C11) instead of the Fab′2 fragment of the anti-CD3 antibody (UCHT1 clone).

FIG. 9B: The cell-killing effect of the unstimulated lymphocyte mediated by the multicellular targeting liposome on target cells; the effector cells are unactivated human lymphocytes, and the target cells are CD19 +Human B lymphocytic leukemia SUP-B15 cells; the cytotoxicity 24 h after dosing is detected by LDH method.

FIG. 9A: The cell-killing effect of the unstimulated lymphocyte mediated by the multicellular targeting liposome on target cells; the effector cells are unactivated CD4+T lymphocytes, and the target cells are LGRS+ human non-small cell lung cancer A549 cells; the cytotoxicity 5 h after dosing is detected by LDH method; the auxiliary antibody of the multicellular targeting liposome is the LGR5 antibody.

FIG. 10: Secretion of IFN-γ under the cell-killing effect of the unstimulated lymphocyte mediated by the multicellular targeting liposome on target cells Raji cells; the effector cells are unactivated human lymphocytes, P×P refers to the non-targeting liposome group, H×P refers to the CD19 single-targeting liposome group, U×P refers to the CD3 single-targeting liposome group, U×H refers to the CD19 and CD3 multicellular targeting liposome group.

FIG. 11: Under the cell-killing effect of the unstimulated lymphocyte mediated by the multicellular targeting liposome on target cells Raji, the impact of different concentrations of EGTA; the effector cells are unstimulated human lymphocytes.

FIG. 12: The cell-killing effect of the unstimulated lymphocyte mediated by the multicellular targeting liposome on target cells; the effector cells are unstimulated human lymphocytes, and the target cells are CD19+ Raji cells; the cytotoxicity 24 h after dosing is detected by LDH method; U×H-lipo refers to the multicellular targeting blank liposome group, U×H-DAS-lipo refers to the multicellular targeting dasatinib liposome group.

### DETAILED DESCRIPTION OF THE PREFERRED EMBODIMENTS

Liposomes are artificially prepared bilayer vesicles formed by lipid molecules. The structure of a lipid molecule usually consists of a hydrophilic head, a hydrophobic tail, and a link section. In the aqueous medium, due to hydrophobic interactions, the hydrophobic tails of the lipid molecules are close to each other, and the hydrophilic heads are arranged facing toward the aqueous phase, forming a bilayer. Based on such a structure, hydrophilic drugs can be embedded in the internal aqueous phase (internal water phase) of the liposome, and hydrophobic drugs can be embedded in the lipid bilayer.

The targeting effect of the targeting liposome is achieved by modifying the ligand on the surface. The ligand may be an antibody, a polypeptide, a small molecule, or the like. The ligand interacts with the target cell specific receptor. The antibody and antibody fragments are modified on the surface of the liposome, and have better binding activity and specificity compared with small molecules and polypeptide ligands. Applying the principle of self-assembly of lipid molecules, it is also feasible to modify two or more different ligands on the surface of one liposome.

Based on the antibody-modified liposome technology, the present disclosure, on the one hand, obtains a novel and unique killing mechanism and effect of liposome on tumor cells through a clever combination of a predominant antibody against a target cell and an auxiliary antibody against an effector cell. The currently known multi-targeting liposome preparation technologies use the combination of different ligands to combine different antigens, to improve the targeting delivery efficiency and selectivity of specific cells. The multicellular targeting liposome of the present disclosure regulates physiological functions such as recognition, binding, and activation between different cells by promoting or blocking interactions between various cells. Therefore, the present disclosure provides a new direction for ligand-targeting liposome technology.

On the other hand, constructing multicellular targeting ability on the surface of a lipid drug carrier is not only more convenient than constructing two antibody sequences with different targeting ability on the same antibody structure, but also can optimize the anti-tumor effect by adjusting the density and proportion of the predominant antibody and auxiliary antibody on the surface of the liposome. In addition, such construction and preparation of the multicellular targeting liposome would also avoid problems such as antibody mismatch and overcome the defects in production and efficacy. In particular, the liposome has unique pharmacokinetic and tissue distribution characteristics in vivo, which greatly improve the defects of short in-vivo half-life of current multi-targeting antibodies.

Further, the multicellular targeting liposome obtained by the present disclosure may combine with liposome drug-loading technology, which is more convenient, diverse and controllable compared with the traditional clinically available combination therapy of multi-targeting antibody and other drugs. By encapsulating the drug in the liposome, the targeting delivery and release of the drug is achieved, that is, the selectivity of the drug is improved, and the releasing site and time of the drug can be changed. For example, the adverse effects of tumor immunotherapy include the production of cytokine storms in patients, so patients are clinically required to take dexamethasone in advance to suppress the adverse reaction. For another example, some immune drugs require systemic administration of cytokine drugs such as IL-2 to achieve the desired immune activation effect. Liposomes, as drug delivery carriers, can achieve targeting delivery of drugs by encapsulating drugs and using predominant antibodies and auxiliary antibodies.

Therefore, by preparing a multicellular targeting liposome including the predominant antibody against the target cell and the auxiliary antibody against the effector cell, the present disclosure can promote the immune cells to bind and recognize tumor cells, and then activate the effector function of the immune cells. The multicellular targeting liposome has a unique mechanism of in-vivo action, and has substantial progress in production, preparation and in-vivo effects compared with the clinically applied multi-targeting antibodies. In addition, the drug-loading liposome may be used to prepare multicellular targeting preparations to achieve better therapeutic effects.

As exemplified in some embodiments of the present disclosure, the technical solution of the present disclosure has the following advantages:

(1) the present disclosure starts from the preparation of a multicellular targeting liposome, modifying two targeting antibodies or antibody fragments with different specificities on the same liposome. The liposome preparation and surface targeting modification are mature in technology, simple in design, and are easy for large-scale production and isolation and purification. The targeting group may be adjusted according to clinical needs, which is conducive to the development and design of various multicellular targeting combinations with different specificities according to the disease.

(2) two antibody fragment-lipid linkers specifically binding to CD19 antigen and CD3 antigen, and the multicellular targeting liposome and single-targeting liposome prepared with fluorescently labeled liposomes show strong binding activity when incubating with CD19 positive and CD3 positive cells, respectively, and such binding is specific;

(3) the multicellular targeting liposomes specifically binding to the CD19 antigen and CD3 antigen are co-incubated with CD19-positive and CD3-positive cells with different fluorescent labels, and two kinds of fluorescent double-positive signals are detected by flow cytometry, showing that multicellular targeting liposomes can effectively mediate the intercellular binding, i.e. the ability to promote immune effector cells to recognize the target cells;

(4) the multicellular targeting liposomes specifically binding to the CD19 antigen and CD3 antigen are co-incubated with immune effector cells and tumor cell, the results show that the immune effector cells have a strong ability to kill tumor cells, and this killing is mediated by the perforin/granzyme pathway;

(5) the multicellular targeting liposomes specifically binding to the CD19 antigen and CD3 antigen are co-incubated with immune effector cells and tumor cell, the ELISA results show that multicellular targeting liposomes can effectively stimulate the secretion of IFN-γ cytokines from immune effector cells;

(6) according to LDH test, the multicellular targeting liposomes specifically binding to the CD19 antigen and CD3 antigen show that the multicellular targeting liposomes have the controllable ability to regulate the cell interaction by regulating the density of the antibodies on the surface;

(7) The multicellular targeting liposomes specifically binding to the CD19 antigen and CD3 antigen not only have the advantages of multi-targeting antibodies, but also can use drug-loading liposomes to prepare multicellular targeting drug delivery systems. By using the two antibody fragment-lipid linkers that specifically bind to CD19 antigen and CD3 antigen and drug-loading liposome loaded with immunoregulatory drug dasatinib as the raw materials, the prepared drug-loading multicellular targeting liposomes show effective inhibiting capacity against immune effector-mediated cell killing under high drug loading. The drug-loading multicellular targeting liposomes may serve as targeting drug carriers to develop into targeting drug delivery systems for diseases and achieve better therapeutic effects.

Before further describing the specific embodiments of the present disclosure, it should be understood that the scope of protection of the present disclosure is not limited to the following specific embodiments; it should also be understood that the terms used in the embodiments of the present disclosure are just for describing the specific embodiments instead of limiting the scope of the present disclosure. The test methods without specific conditions noted in the following embodiments are generally based on conventional conditions or the conditions recommended by the manufacturers.

When the numerical values are given by the embodiments, it is to be understood that the two endpoints of each numerical range and any one between the two may be selected unless otherwise stated. Unless otherwise defined, all technical and scientific terms used in the present disclosure have the same meaning as commonly understood by one skilled in the art. In addition to the specific method, equipment and material used in the embodiments, any method, equipment and material in the existing technology similar or equivalent to the method, equipment and material mentioned in the embodiments of the present disclosure may be used to realize the invention according to the grasp of the existing technology and the record of the invention by those skilled in the art.

Unless otherwise stated, the experimental methods, detection methods, and preparation methods disclosed in the present invention all employ conventional techniques of molecular biology, biochemistry, chromatin structure and analysis, analytical chemistry, cell culture, recombinant DNA technology in the technical field and related fields. These techniques are well described in the prior literature. For details, please see Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third Edition, Academic Press, San Diego, 1998; METHOD IN ENZYMOLOGY, Vol. 304, Chromatin (PMWassarman and APWolffe, eds.), Academic Press, San Diego, 1999; and METHODS IN MOLECULAR BIOLOGY, Vol. 119, Chromatin Protocols (PBBecker, ed.) Humana Press, Totowa, 1999, etc.

Unless otherwise specified, the experimental materials used in the following embodiments were purchased from conventional biochemical reagent stores.

### Experimental Reagents and Cells

Lecithin (EPC), hydrogenated soybean phosphatidylcholine (HSPC) and distearoyl phosphatidylethanolamine-polyethylene glycol 2000-maleimide (DSPE-PEG2000-mal) were purchased from NOF Corporation (Tokyo, Japan). Cholesterol, distearoyl phosphatidylethanolamine-polyethylene glycol 2000 (DSPE-PEG2000) were purchased from Avanti Polar Lipids (AL, US).

DiI was purchased from Sigma and CFSE was purchased from Invitrogen.

RPMI-1640 medium, fetal calf serum, penicillin-streptomycin double-antibody reagent (100×) were purchased from Gibico.

Raji human lymphoma cells and Jurkat human lymphoma cells were purchased from the Institute of Cells, Chinese Academy of Sciences.

### EMBODIMENT 1

### Preparation of Multicellular Targeting Liposome

### 1. Preparation of F(ab′)2 Fragments of Anti-CD19 Antibody and Anti-CD3 Antibody

The F(ab′)2 fragment is prepared by excising the Fc fragment of IgG by the digestion reaction of an immobilized enzyme (pepsin or ficin). The human IgG and mouse IgG are digested by pepsin, and mouse IgG1 is digested by ficin. The specific steps are as follows: centrifuging 500 μl of antibody, and passing through a desalting column; adding the flow-through liquid to the immobilized enzyme, and mixing at 37° C.; incubating the pepsin for 4 h or incubating the ficin for 24 h, collecting the flow-through liquid by centrifuging at 5,000 g for 1 min, washing the immobilized enzyme with Protein A Binding Buffer and collecting the flow-through liquid, and merging the flow-through liquids to obtain the enzyme-digested product.

Mixing the enzyme-digested product at a constant temperature in Protein A column for 10 min, collecting the flow-through liquid by centrifuging, washing the protein A column with Protein A Binding Buffer and collecting the flow-through liquid. Merging the flow-through liquids to obtain the enzyme-digested product purified by Protein A column and with the large fragments of IgG and Fc removed. Dialyzing the above enzyme-digested product through a 50 KD dialysis bag, and the dialysis medium is PBS solution (pH=7.0). The dialysis is performed in the order of 2 h, 2 h and 16h to obtain a purified antibody F(ab′)2 fragment with the Fc fragment removed.

It is confirmed by the traditional technology that the obtained anti-CD19 antibody F(ab′)2 fragment is the full-length anti-CD19 antibody with Fc fragment being removed.

It is confirmed by the traditional technology that the obtained anti-CD3 antibody F(ab′)2 fragment is the full-length anti-CD3 antibody with the Fc fragment being removed. A light chain variable region and a heavy chain variable region are included. The amino acid sequence of the light chain variable region is shown in SEQ. ID. NO. 1, specifically:

DIQMTQTTSSLSASLGDRVTISCRASQDIRNYLNWYQQKPDGTVKLLIYYT
SRLHSGVPSKFSGSGSGTDYSLTISNLEQEDIATYFCQQGNTLPWTFAGGT
KLEIK.

The amino acid sequence of the heavy chain variable region is shown in SEQ ID NO. 2, specifically:

EVQLQQSGPELVKPGASMKISCKASGYSFTGYTMNWVKQSHGKNLEWMGLI
NPYKGVSTYNQKFKDKATLTVDKSSSTAYMELLSLTSEDSAVYYCARSGYY
GDSDWYFDVWGQGTTLTVFS.

### 2. Preparation Fab′ Fragment (of Anti-CD19 Antibody and Anti-CD3 Antibody)-Polymer-Lipid Linker

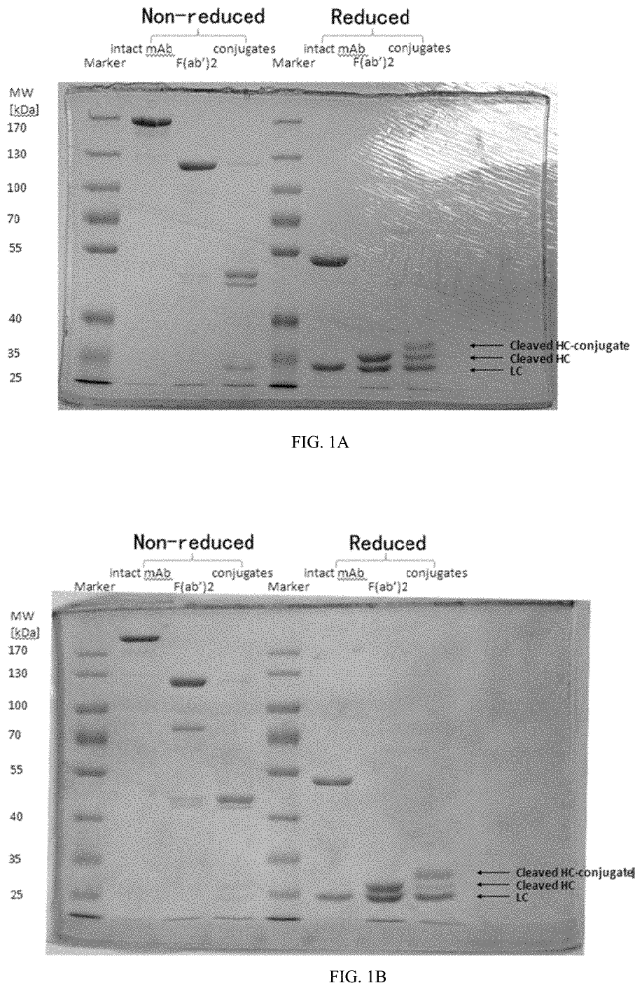

The antibody F(ab′)2 fragment is reduced to Fab′ fragment by using β-mercaptoethylamine as a reducing agent. The Fab′ fragment is chemically linked to the maleimide group of DSPE-PEG2000-Mal. The specific steps are as follows: mixing the F(ab′)2 fragment (1-10 mg/ml) with 50 mM EDTA-containing β-mercaptoethylamine, reacting at 37° C. under the protection of N₂ with shaking for 90 min; centrifuging, passing through a desalting column and collecting the flow-through liquid. Fab′, the reduction product of the antibody F(ab′)2 fragment from which β-mercaptoethylamine is removed, is obtained. Then, adding 30 mM HEPES solution containing 600 uM DSPE-PEG2000-Mal micelle, so that the molar ratio of Fab′ to DSPE-PEG2000-Mal is 1:1, and the reaction is performed at 10° C. under the protection of N₂ with shaking for 16 h. The antibody ab′ fragment-polymer-lipid linker, i.e. DSPE-PEG2000-Mal-Fab′ micelle, is obtained. The linking situation of the obtained linker product is examined by SDS-PAGE. The results are shown in FIGS. 1A and 1B.

3. Preparation of Anti-CD3 Antibody scFv Fragment-Polymer-Lipid Linker

The antibody scFv fragment and its multimer are reduced to scFv containing free sulfydryl group by using tricarboxyethyl phosphine (TCEP) as a reducing agent, and chemically linked to the maleimide group of DSPE-PEG2000-Mal. The specific steps are as follows: mixing 800 μl of scFv fragment (1-10 mg/ml) with HEPES solution containing 1.2 mM TCEP, so that the molar ratio of scFv to TCEP is 1:5, then adding 30mM HEPES solution containing 600 μM DSPE-PEG2000-Mal micelle immediately, and reacting at 10° C. under the protection of N₂ with shaking for 16 h; The antibody scFv fragment-polymer-lipid linker, i.e. DSPE-PEG2000-Mal-scFv micelle, is obtained. The linking situation of the obtained linker product is examined by SDS-PAGE. The result is shown in FIG. 1C.

### 4. Preparation of Anti-CD19 and Anti-CD3 Single-Targeting and Multicellular Targeting Liposomes

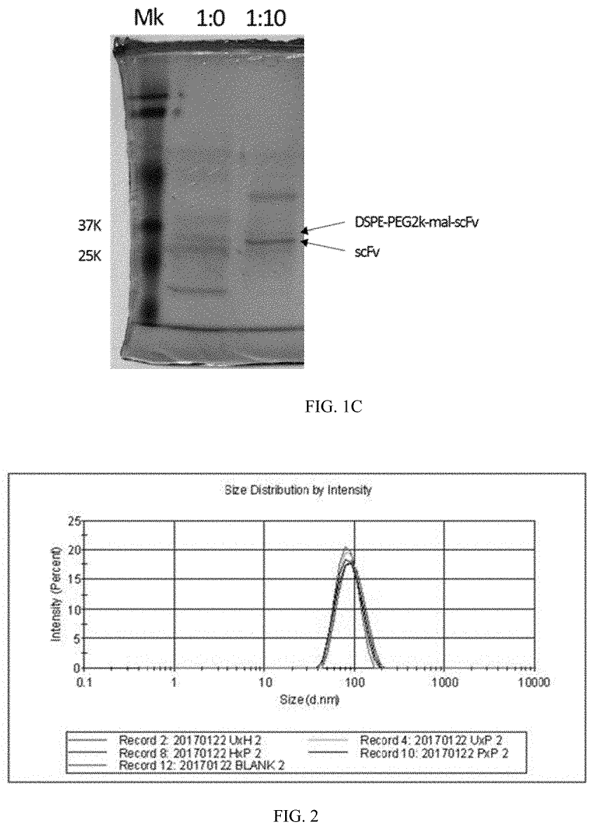

The single-targeting and multicellular targeting liposomes are prepared by the post-insertion method. For multicellular targeting liposome, the prepared anti-CD19 and anti-CD3 Fab′ fragment-polymer-lipid linker (DSPE-PEG2000-Mal- Fab′) or scFv fragment-polymer-lipid linker is mixed with liposome in a molar ratio (0.1-5):(0.1-5):1000, and the reaction is performed at 37° C. under the protection of N₂ with shaking for 24 h. Then, the liposomes are washed 5 times by 300 KD ultrafiltration to remove the unlinked antibody Fab′ fragments to obtain anti-CD19 and anti-CD3 multicellular targeting liposomes. For anti-CD3 single-targeting liposomes, the anti-CD19 Fab′ fragment-polymer-lipid linker DSPE-PEG2000-Mal-Fab′ is replaced with equimolar DSPE-mPEG2000. For anti-CD19 single-targeting liposomes, the anti-CD3 Fab′ fragment-polymer-lipid linker DSPE-PEG2000-Mal-Fab′ is replaced with equimolar DSPE-mPEG2000. The results are shown in FIG. 2 and Table 1: The particle size of the prepared targeting liposomes is about 80-90 nm, and the PDI is about 0.1.

TABLE 1

| | Z-AVE (nm) | PDI |
|---|---|---|
| blank | 80.3 ± 1.5 | 0.06 ± 0.01 |
| aCD3 × aCD19 | 89.0 ± 2.3 | 0.08 ± 0.01 |
| aCD3 × mPEG | 84.1 ± 3.2 | 0.11 ± 0.04 |
| aCD19 × mPEG | 83.2 ± 0.8 | 0.08 ± 0.02 |
| mPEG × mPEG | 82.8 ± 3.6 | 0.10 ± 0.01 |

### EMBODIMENT 2

### Evaluation of in-vitro Specific Binding Effect of Multicellular Targeting Liposome

CD19-positive Raji cells and CD3-positive Jurkat cells are used as representatives to investigate the in-vitro specific binding effect of multicellular targeting liposomes.

Jurkat, Clone E6-1 human T lymphocytic leukemia cell line and Raji human B cell lymphoma cell line were purchased from the Cell Bank of the Chinese Academy of Sciences. The cell strain passes through a complete medium of RPMI-1640 (GIBCO) containing 1% pls double antibody +10% FBS.

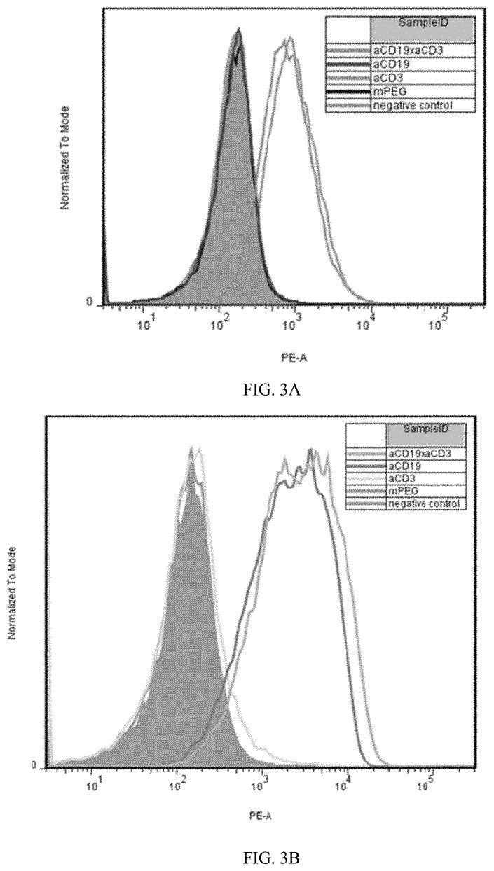

CD3-positive cell line Jurkat cells, CD19-positive cell line Raji cells, and human peripheral blood lymphocytes are analyzed by flow cytometry to examine the in-vitro specific binding capacity of the multicellular targeting liposome. 1×10⁶ Raji or Jurkat cells are resuspended in 400 μl complete medium, added to a 48-well plate, with lug single-targeting DiI labeled liposomes or multicellular targeting DiI labeled liposomes added to each well. Incubating in a cell incubator at 37° C. for 2 h. Then removing the cells and placing in a flow tube, centrifuging at 500×g for 5 min, discarding the supernatant, and washing the cells twice with PBS. Resuspending the cells with 400 μl PBS and detecting the sample by flow cytometry Becton Dickinson FACS LSRII. The results are shown in FIGS. 3A and 3B.

### EMBODIMENT 3

### Evaluation of Intercellular Binding Effect of Target Cells Mediated by Multicellular Targeting Liposome

CD19-positive Raji cells and CD3-positive Jurkat cells are used as representatives to demonstrate that the multicellular targeting liposome can mediate the cell-cell binding between different target cells.

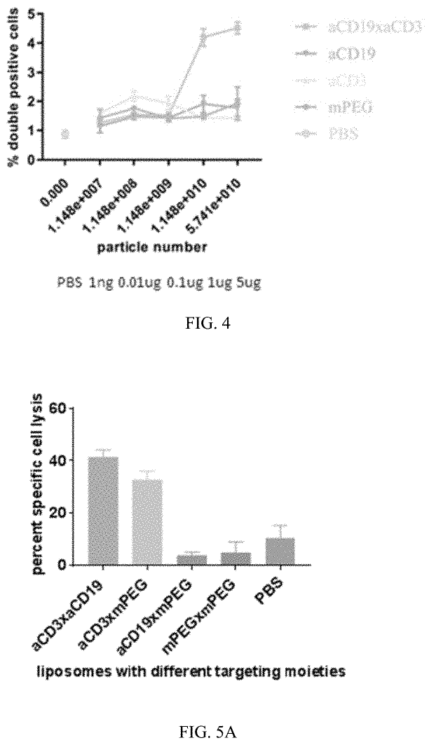

Jurkat cells are stained by CFSE, and Raji cells are stained by DiI. 1.25×10⁵ Raji-DiI and 1.25×10⁵ Jurkat-CFSE cells are resuspended in 400 μl phenol red-free complete medium, mixed and added to a 48-well plate, with 1 μg single-targeting liposomes or multicellular targeting liposomes added to each well. Incubating in a 37° C. cell incubator for 30 min. CFSE-positive Jurkat cells, DiI-positive Raji cells, and CFSE/DiI double-positive cell clusters are analyzed by flow cytometry to examine the intercellular binding effect of target cells mediated by multicellular targeting liposomes. The result is shown in FIG. 4.

### EMBODIMENT 4

### Evaluation of the Killing Effect of Effector Cells Mediated by Multicellular Targeting Liposome on Tumor Cells

The cell culture medium is formulated by adding 5% FBS, 1% double antibody, and 100 IU/ml hIL-2 to phenol red-free RPMI-1640 medium.

### 1. Evaluation of Tumor Cell Killing Effect by LDH Method

The killing effect of effector cells mediated by multicellular targeting liposome on tumor cells is detected by CytoTox 96® non-radioactive cytotoxicity test kit (Promega Co.).

Effector cells (unstimulated human lymphocytes, CD3/CD28 antibody amplified and activated human T cells, γδ T cells, LAK cells) and target cells (Raji cells, SUP-B15 cells, A549 cells) are resuspended in phenol red-free complete medium, and the cell concentrations are 1*10\hat{ }6 cells/ml and 1.11*10\hat{ }5 cells/ml, respectively. Adding 100 μl effector cells and 90 μl target cells to a 96-well plate and mixing by pipetting to plate the cells. At this time, the ratio of target cells to effector cells is 1:10. Adding 10 μl of targeting liposomes of different concentrations to each well, and mixing by pipetting. Placing the 96-well plate in a cell incubator, incubating for 24 h. 45 min before the end of the incubation, adding 10 μl lysate to the volume-correcting control well and the targeting cell maximum lysis well. After the incubation, centrifuging the 96-well plate at 250 g for 4 min. After centrifugation, pipetting 50 μl of the supernatant to a new 96-well plate, adding 50 μl of LDH substrate to the new well plate, and incubating at 22° C. for 15 min. After incubation, detecting the absorbance in a microplate reader at a wavelength of 490 nm.

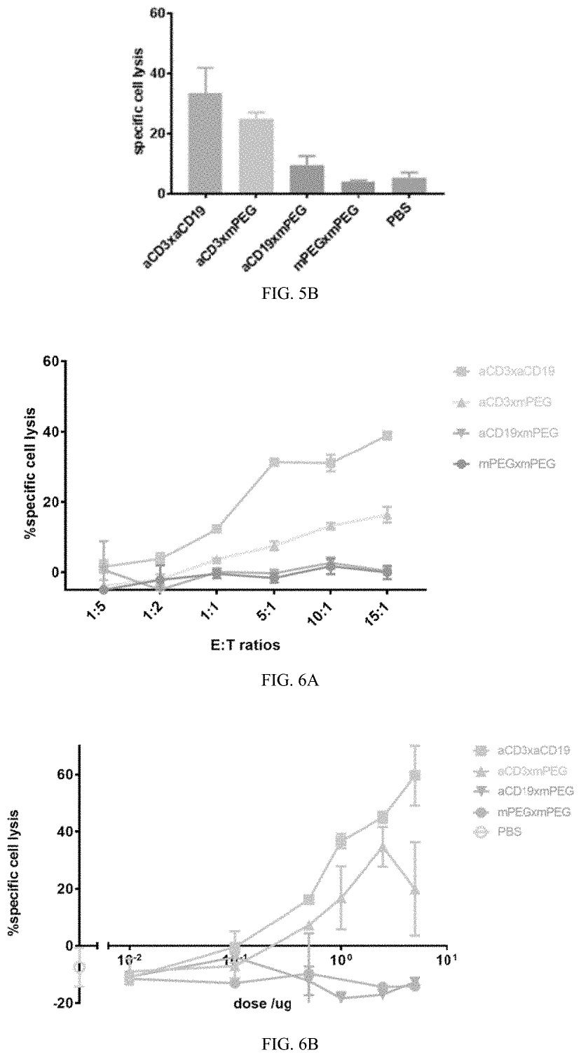

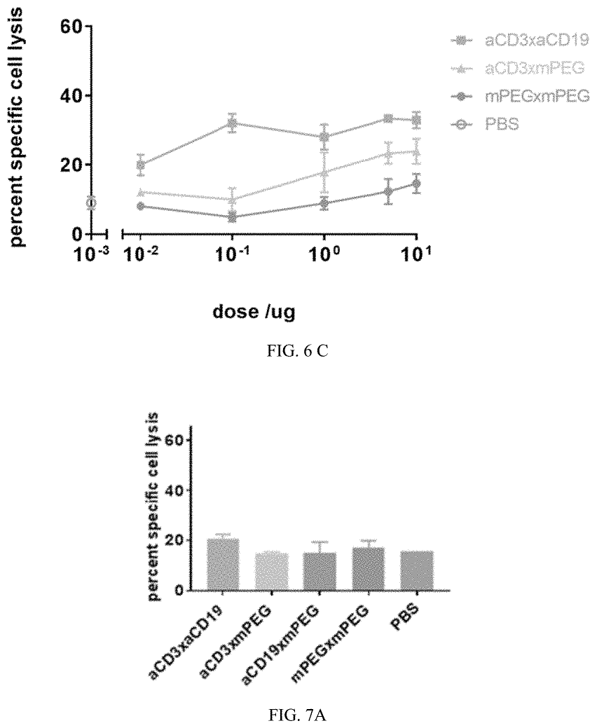

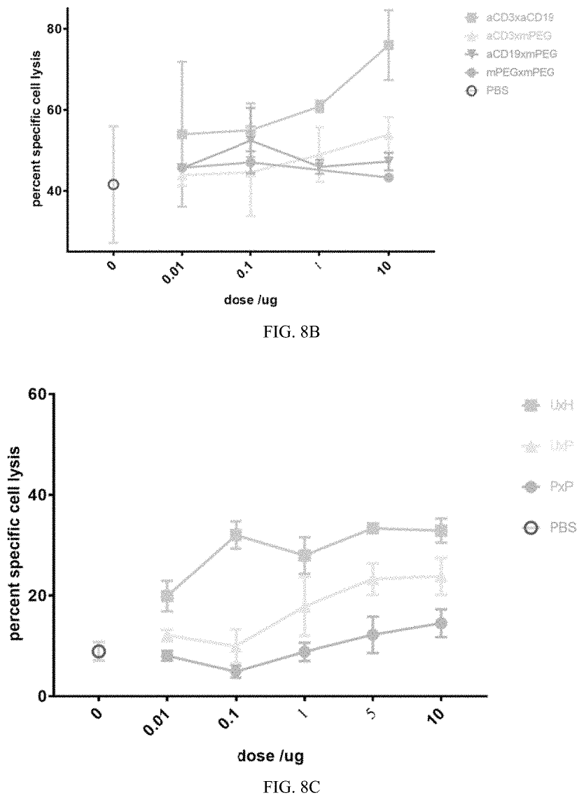

The results are shown in FIGS. 5A, 5B, 6A, 6B, 6C, 8B, 8C, and 9.

These experimental data fully demonstrate that the multicellular targeting liposome of the present disclosure can effectively mediate the effector cells to effectively kill the target cells, and the effector cells may include unstimulated lymphocytes, in-vitro amplified lymphocytes, in-vitro activated lymphocytes, in-vitro induced differentiated lymphocytes. The cytotoxic effect is dependent on cell lysis and is dose-dependent.

### 2. Evaluation of Tumor Cell Killing Effect by PI Staining Method

Effector cells (unstimulated human lymphocytes, CD3/CD28 antibody amplified and activated human T cells, γδ T cells, LAK cells) and CFSE-labeled target cells (Raji cells) are resuspended in phenol red-free complete medium, and the cell concentrations are 1*10\hat{ }6 cells/ml and 1.11*10\hat{ }5 cells/ml, respectively. Adding 100 μl effector cells and 90 μl target cells to a 96-well plate and mixing by pipetting to plate the cells. At this time, the ratios of target cells to effector cells are 1:1, 1:5, and 1:10. Adding 10 μl of targeting liposomes of different concentrations to each well, and mixing by pipetting. Place the 96-well plate in a cell culture incubator for 24 h. After the incubation, collecting the cell suspension into a flow tube, adding 5 μmg/ml PI, incubating for 15 min, then adding 200 μl of flow buffer and loading the sample.

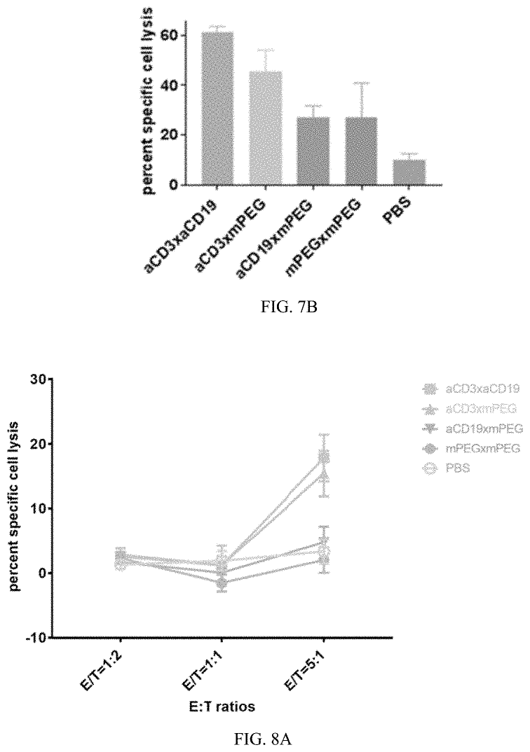

The results are shown in FIGS. 7A, 7B, 8A and 9B.

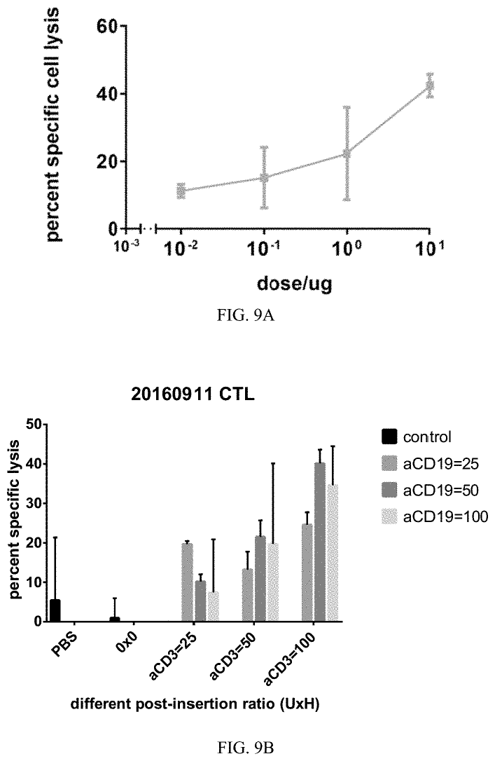

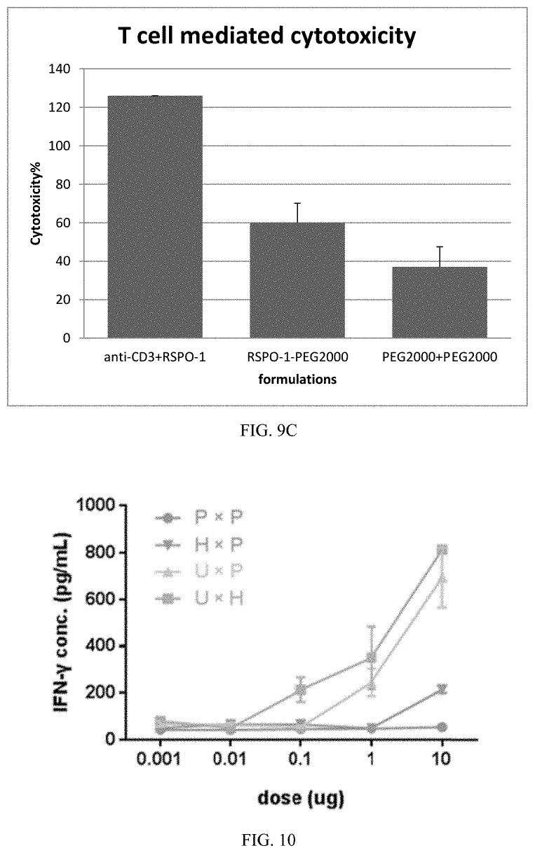

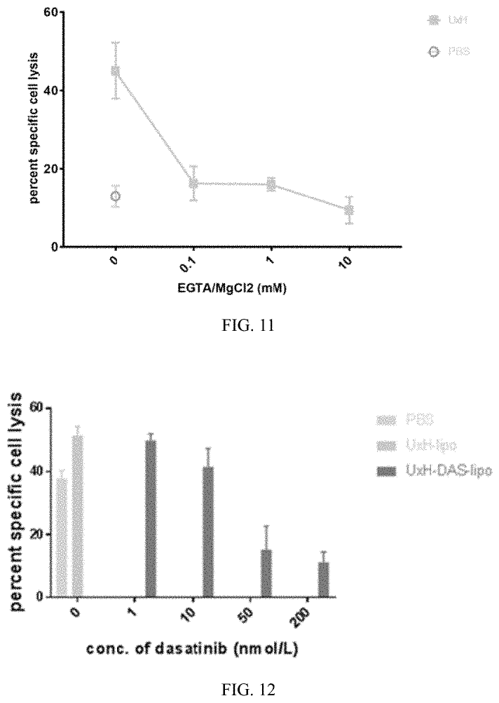

These experimental data fully demonstrate that the multicellular targeting liposome of the present disclosure can effectively mediate the effector cells to effectively kill the target cells, and the effector cells may include unstimulated lymphocytes, in-vitro amplified lymphocytes, in-vitro activated lymphocytes, in-vitro differentiated lymphocytes. The cytotoxic effect is dependent on the membrane lysis effect of cell lysis, and is dose-dependent. In addition, the multi-targeting antibodies can effectively mediate the killing of effector cells against different target cells expressing the same antigen (FIG. 9A CD19 +SUP-B15 cells).

The results of replacing different antibody molecules or target cells are shown in FIGS. 9A, 9B, and 9C. The predominant antibody of FIG. 9A is replaced with anti-CD3 antibodies of different clones (2C11) and different antibody forms (IgG). FIG. 9B is a multicellular targeting liposome with the same predominant antibody and auxiliary antibody, while the target cells are replaced with CD19+ Human leukemia SUP-B15 cells. The auxiliary antibody of FIG. 9C is replaced with a different targeting molecule (RSPO-1 protein) that targets a different antigen (LGR), and the target cells are replaced with non-small cell lung cancer A549 cells (solid tumor model).

These experimental data fully demonstrate that the first antibody and auxiliary antibody of the multicellular targeting liposome of the present disclosure may be a targeting molecule of any form and structure that targets any antigen, and the target cell and effector cell may be any cell that the predominant antibody and auxiliary antibody can bind to. The multicellular targeting liposome mediates the interaction between immune effector cells and target cells based on the targeting ability. According to the types of different diseases, we may effectively mediate the effect of effector cells on target cells by replacing any targeting molecules (predominant antibody and auxiliary antibody).

### EMBODIMENT 5

### Evaluation of the Activation Effect of Multicellular Targeting Liposome Stimulating Effector Cells

Effector cells (unstimulated human lymphocytes) and target cells (Raji cells) are resuspended in phenol red-free complete medium, and the cell concentrations are 1*10^6 cells/ml and 1.11*10\hat{ }5 cells/ml, respectively. Adding 100 μl effector cells and 90 μl target cells to a 96-well plate and mixing by pipetting to plate the cells. At this time, the ratio of target cells to effector cells is 10:1. Adding 10 μl of targeting liposomes of different concentrations to each well, and mixing by pipetting. Placing the 96-well plate in a cell incubator, incubating for 24 h. After the incubation, centrifuging the 96-well plate at 250 g for 4 min. After centrifugation, pipetting 50 μl of the supernatant to a new 96-well plate. Detecting the IFN-y in the supernatant by ELISA method. The result shown in FIG. 10 fully demonstrates that the multicellular targeting liposome of the present disclosure can effectively mediate the unstimulated lymphocytes, in-vitro amplified lymphocytes, in-vitro activated lymphocytes, in-vitro differentiated lymphocytest to effectively kill the target cells, while promoting the activation of effector cells, the production and release of cytokines. The produced cytokines have anti-tumor activity and is dose-dependent.

### EMBODIMENT 6

### Study on the Mechanism of Multicellular Targeting Liposome

Effector cells (unstimulated human lymphocytes) and target cells (Raji cells) are resuspended in phenol red-free complete medium, and the cell concentrations are 1.11*10\hat{ }6 cells/ml and 1.11*10\hat{ }5 cells/ml, respectively. Adding 90 μl effector cells and 90 μl target cells to a 96-well plate and mixing by pipetting to plate the cells. At this time, the ratio of target cells to effector cells is 10:1. Adding 10 μl of multicellular targeting liposome to each well, and mixing by pipetting. Adding 10 μl A EGTA/MgCl₂ to each well with the final concentration of 0 mM, 0.1 mM, 1 mM and 10 mM, and mixing by pipetting. Placing the 96-well plate in a cell incubator, incubating for 24 h. 45 min before the end of the incubation, adding 10 μL lysate to the volume-correcting control well and the targeting cell maximum lysis well. After the incubation, centrifuging the 96-well plate at 250 g for 4 min. After centrifugation, pipetting 50 of the supernatant to a new 96-well plate, adding 50 μl of LDH substrate to the new well plate, and incubating at 22° C. for 15 min. After incubation, detecting the absorbance in a microplate reader at a wavelength of 490 nm.

The result shown in FIG. 10 fully demonstrates that the killing effect of the effector cells mediated by the multicellular targeting liposome on target cells is based on the calcium ion-dependent granzyme/perform pathway of the effector cells.

### EMBODIMENT 7

### Evaluation of Targeting Drug Delivery Effect of Multicellular Targeting Liposome

The drug-loading multicellular targeting liposomes are prepared by the post-insertion method. For multicellular targeting drug-loading liposome, the prepared anti-CD19 and anti-CD3 Fab′ fragment-polymer-lipid linker (DSPE-PEG2000-Mal-Fab′) is mixed with the dasatinib drug-loading liposome in a molar ratio (0.1-5): (0.1-5): 1000, and the reaction is performed at 37 ° C. under the protection of N₂ with shaking for 24 h. Then, the liposomes are washed 5 times by 300 KD ultrafiltration to remove the unlinked antibody Fab′ fragments to obtain the multicellular targeting dasatinib liposome.

Effector cells (unstimulated lymphocytes) and target cells (Raji cells) are resuspended in phenol red-free complete medium, and the cell concentrations are 1*10\hat{ }6 cells/ml and 1.11*10\hat{ }5 cells/ml, respectively. Adding 100 μl effector cells and 90 μl target cells to a 96-well plate and mixing by pipetting to plate the cells. At this time, the ratio of target cells to effector cells is 1:10. Adding 10 μl of multicellular targeting drug-loading liposomes with different dasatinib concentrations and same lipid concentrations to each well, and mixing by pipetting. Placing the 96-well plate in a cell incubator, incubating for 24 h. 45 min before the end of the incubation, adding 10 μl lysate to the volume-correcting control well and the targeting cell maximum lysis well. After the incubation, centrifuging the 96-well plate at 250 g for 4 min. After centrifugation, pipetting 50 μl of the supernatant to a new 96-well plate, adding 50 μl of LDH substrate to the new well plate, and incubating at 22° C. for 15 min. After incubation, detecting the absorbance in a microplate reader at a wavelength of 490 nm.

The result shown in FIG. 10 fully demonstrates that by linking the predominant antibody and the auxiliary antibody to the drug-loading liposome, the drug-loading effect can be fully exerted on the basis of the multicellular targeting mechanism.

The above are only some preferred embodiments of the present disclosure instead of limitations on the present disclosure in any form or substance. It should be noted that, for those skilled in the art, improvements and supplements may be made without departing from the method of the present disclosure, the improvements and supplements shall also be covered by the protection of the present disclosure. The equivalent changes of alternations, modifications and evolutions can be made by those skilled in the art using the technical contents revealed above and without departing from the spirit and scope of the present disclosure, and those equivalent changes are regarded as equivalent embodiments of the present disclosure. Meanwhile, any alterations, modifications, and evolutions of any equivalent changes made to the above embodiments according to the essential technology of the present disclosure still fall within the scope of the technical solution of the present disclosure.

1. A multicellular targeting liposome, comprising a liposome, and a predominant antibody and an auxiliary antibody modified on a surface of the liposome, wherein the predominant antibody specifically binds to a target molecule on a surface of a target cell, and the auxiliary antibody specifically binds to an immune effector cell.

2. The multicellular targeting liposome according to claim 1, wherein the multicellular targeting liposome is capable of simultaneously or sequentially binding to the immune effector cell and the target cell, such that the immune effector cell recognizes the target cell and activates the immune effect.

3. The multicellular targeting liposome according to claim 1, wherein the molar ratio of the predominant antibody to the auxiliary antibody on each liposome is between 100/1 and 1/1.

4. The multicellular targeting liposome according to claim 1, wherein each liposome has 1-1000 predominant antibodies and 1-1000 auxiliary antibodies on the surface.

5. The multicellular targeting liposome according to claim 1, wherein the predominant antibody and the auxiliary antibody are displayed on the surface of the liposome through covalent linkage or hydrophobic, hydrophilic interaction, respectively.

6. The multicellular targeting liposome according to claim 1, wherein an antigen targeted by the predominant antibody is selected from a group consisting of a microbial antigen, a tumor-related antigen, a tumor cell surface specific antigen, a highly expressed antigen on a tumor cell surface, and a highly expressed antigen in a tumor tissue or a tumor blood vessel; an antigen targeted by the auxiliary antibody is an antigen expressed by a tumor cell, a lymphocyte or a myeloid cell.

7. The multicellular targeting liposome according to claim 1, which is capable of simultaneously or sequentially binding to a tumor cell, a lymphocyte, or a myeloid cell.

8. The multicellular targeting liposome according to claim 1, wherein the multicellular targeting liposome targets and binds to a T lymphocyte, an NK cell, an NKT cell, a macrophage, or a neutrophil.

9. The multicellular targeting liposome according to claim 1, wherein the multicellular targeting liposome targets and binds to an unactivated T cell, or an in-vitro activated or amplified T cell.

10. The multicellular targeting liposome according to claim 9, wherein the T lymphocyte is selected from a group consisting of an antigen-specific T lymphocyte, a tumor infiltrating lymphocyte, a cytotoxic T cell, a helper T cell, a lymphokine-activated killer cell, a γδ T cell, a chimeric antigen receptor T cell, and a T cell receptor chimeric T cell.

11. The multicellular targeting liposome according to claim 1, wherein a form of the predominant antibody and the auxiliary antibody is selected from a group consisting of IgG, Fab′ fragment, F(ab′)2 fragment, Fab fragment, single-chain Fv fragment, minibody, nanobody, unibody, and diabody.

12. The multicellular targeting liposome according to claim 1, wherein an antigen targeted by the primary antibody is selected from a group consisting of CD19, CD20, PSMA, Her2/neu, EGFR and LGR5.

13. The multicellular targeting liposome according to claim 1, wherein the auxiliary antibody is selected from a group consisting of CD3 antibody and a fragment, a PD1 antibody and a fragment, a CTLA4 antibody and a fragment, a CD40L antibody and a fragment, or a combination thereof

14. The multicellular targeting liposome according to claim 1, wherein components of the liposome comprise phosphatidylcholine, cholesterol, a lipid linked to the predominant antibody, and a lipid molecule linked to the auxiliary antibody.

15. The multicellular targeting liposome according to claim 1, wherein the liposome is a blank liposome or a drug-loading liposome.

16. A method for preparing the multicellular targeting liposome according to any one of claims 1 to 15, comprising: (1) connecting the predominant antibody and the auxiliary antibody with a lipid molecule respectively to obtain a predominant antibody-lipid molecule and an auxiliary antibody-lipid molecule; (2) mixing the predominant antibody-lipid molecule and the auxiliary antibody-lipid molecule with a constructed liposome, and incubating to obtain a mixed solution; (3) dialyzing or ultrafiltrating the mixed solution to remove unloaded antibody and antibody-lipid complexes, and obtaining the multicellular targeting liposome.

17. Use of the multicellular targeting liposome of any one of claims 1 to 15 in preparing immunotherapeutic drugs and antitumor drugs.

18. A method for treating tumor, comprising: administering the multicellular targeting liposome of any one of claims 1 to 15 to a tumor patient.
